(19) United States
(12) Patent Application Publication
(10) Pub. No.: US 2014/0379630 A1
(43) Pub. Date: Dec. 25, 2014

(54) DISCOVERING ADVERSE HEALTH EVENTS VIA BEHAVIORAL DATA

(71) Applicant: Microsoft Corporation, Redmond, WA (US)

(72) Inventors: Eric J. Horvitz, Kirkland, WA (US); Ryen William White, Redmond, WA (US)

(21) Appl. No.: 13/924,899

(22) Filed: Jun. 24, 2013

(51) Int. Cl. G06F 17/30

(52) U.S. Cl. CPC G06F 17/30592
USPC 707/603

(57) ABSTRACT

Aspects of the subject disclosure are directed towards processing search logs and/or other large scale data sources to detect medical related-effects. For example, an anomalous number of queries regarding a particular symptom and a drug may indicate the existence of a previously unknown side-effect of the drug. Side effects of drug interactions may also be found by processing behavioral data such as queries and social network posts. Also described is the generation of symptom spectra data that is processed to detect anomalies and the like in user behavior corresponding to medical related-effects.

### BACKGROUND

The Food and Drug Administration (FDA) and other organizations collect reports on drug side effects from physicians, pharmacists, patients, and drug companies. These reports provide valuable clues about drug-related adverse events, but are relatively incomplete and are sometimes biased. As a result, adverse event alerts for single drugs are often delayed as evidence accumulates. These challenges are compounded with respect to adverse events resulting from multiple drug interactions.

Adverse drug events cause substantial morbidity and mortality and are often discovered after a drug comes to market. Adverse events are often discovered by chance, although more recently some are identified by mining databases containing self-reported adverse event data. The most popular example of such a system is the adverse event reporting system (AERS) managed by the Food and Drug Administration (FDA). In AERS, patients, physicians, and pharmaceutical companies provide information on interactions between drugs and possible side effects that they believe (based on experience with them) may be caused by the medications. However, because this requires explicit self-reporting, the amount of data that can be gathered in this way is extremely limited. There may also be a significant time lag between side effects being experienced and reporting to AERS or the availability of signals in from other sources such as electronic health records.

### SUMMARY

This Summary is provided to introduce a selection of representative concepts in a simplified form that are further described below in the Detailed Description. This Summary is not intended to identify key features or essential features of the claimed subject matter, nor is it intended to be used in any way that would limit the scope of the claimed subject matter.

Briefly, various aspects of the subject matter described herein are directed towards processing large-scale behavioral data to identify health-related effects in which a target outcome is unknown, including recognizing signals in the large-scale behavioral data. This may include detecting anomalous querying patterns with respect to expected querying patterns, and taking action (e.g., outputting data) upon detecting anomalous querying patterns.

One or more aspects are directed towards an offline data processing subsystem configured to access behavioral data from one or more sources. The subsystem generates symptom or other spectra data based upon the behavioral data, in which the symptom spectra data comprises a probability distribution across a standard set of symptoms computed using different groups of users. An example of spectra other than symptom spectra includes medical conditions.

One or more aspects are directed towards generating a representation of behavioral data with respect to large scale sets of medical entity information. The representation is processed to recognize health-related effects of one or more medical-related entities, in which a target outcome is unknown, based upon statistical analysis.

Other aspects and advantages may become apparent from the following detailed description when taken in conjunction with the drawings.

### BRIEF DESCRIPTION OF THE DRAWINGS

The present invention is illustrated by way of example and not limited in the accompanying figures in which like reference numerals indicate similar elements and in which:

FIG. 1 is a block diagram representing an example system/architecture for using behavioral data to discover health-related events, according to one or more example implementations.

FIG. 2 is an example partial screenshot showing a symptom spectra representation obtained by processing behavioral data, according to one or more example implementations.

FIG. 3 is an example partial screenshot showing a matrix of drug interaction detection computations, according to one or more example implementations.

FIG. 4 is a flow diagram showing example steps that may be taken to discover health-related events from behavioral data, according to one or more example implementations.

FIG. 5 is a block diagram representing example non-limiting networked environments in which various embodiments described herein can be implemented.

FIG. 6 is a block diagram representing an example non-limiting computing system or operating environment in which one or more aspects of various embodiments described herein can be implemented.

### DETAILED DESCRIPTION

Various aspects of the technology described herein are generally directed towards identifying possible adverse medical events (or sometimes beneficial ones) from search log data and other behavioral data sources collected at scale, particularly when the outcome (e.g., symptoms/a certain condition) is not known in advance. In other words, the technology is directed towards discovering side-effects, including from interactions between drugs, devices, procedures, etc., rather than confirming them.

To this end, internet users may provide early (or recently-appearing) clues about adverse drug events via their online information-seeking behaviors recorded by search engines. The technology describe herein identifies pairs (or other tuples) of medications (or other factors such as medical devices or procedures) for further exploration and analysis when the target outcome may not be known a priori and background effects need to be considered.

In one aspect, described is an exploratory analysis based upon exploring the effect of medical-related entities including drugs, medical devices and/or procedures across a broad spectrum of symptoms unrelated to any particular condition. Also described are modeling relations between conditions and symptoms (including potential causal effects). This includes the effect of medications as well as application scenarios such as detecting the impact of medical devices and procedures on symptom searching.

It should be understood that any of the examples herein are non-limiting. For instance, some of the examples herein are directed towards drug interactions, however single drug side-effects may be discovered by the technology herein, as well as side effects related to one or more medical devices and/or medical procedures. As such, the present invention is not limited to any particular embodiments, aspects, concepts, structures, functionalities or examples described herein. Rather, any of the embodiments, aspects, concepts, structures, functionalities or examples described herein are non-limiting, and the present invention may be used various ways that provide benefits and advantages in data mining and data analysis in general.

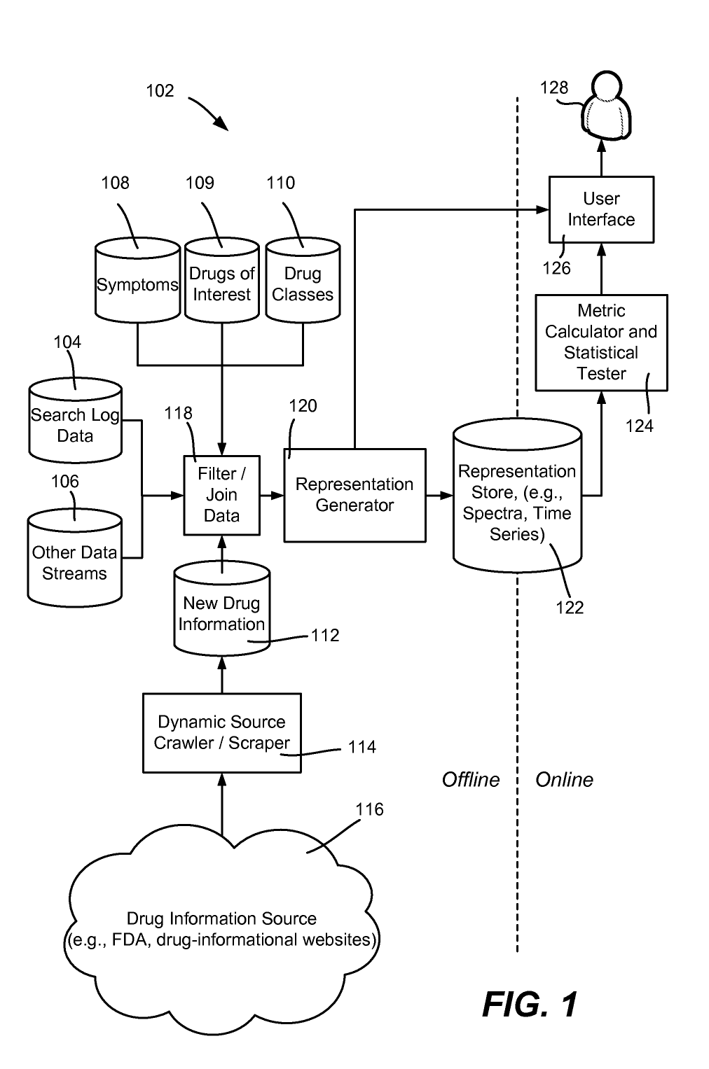

FIG. 1 is a block diagram showing an example architecture of a behavioral log-based adverse event reporting system 102. Various data sources 104, 106 and 108-110 (e.g., including search log data 104 containing queries from many search engines gathered using a browser toolbar or from a particular engine using their server-side logging mechanisms), are mined to find data of people searching for drugs of interest, for example, as well as other information such as the background distribution of symptoms across multiple searchers independent of any drug event. Various data may be collected via server-side instrumentation, browser add-ins, and/or toolbar plug-ins, for example.

As shown in FIG. 1, these data, along with new drug information 112 obtained from a dynamic source crawler/scraper 114 from a suitable source 116 are input into a “Filter/Join Data” phase 118.

The various data are used to generate (block 120) representations of the data such as symptom spectra and (aligned) time series that can be stored in a suitable store 122 and used internally to compute metrics (block 124) as described herein. These representations may be in any data set (e.g., a data structure) including visible representations presented via a user interface 126 to the user 128 (analyst or consumer), such as via a “dashboard” type of interface. Note that in the exemplified implementation shown in FIG. 1, the steps to mine the log data and generate the representations are performed offline across potentially very large volumes of search data.

Once the representations are generated in the offline subsystem, a variety of metrics can be computed from them that capture degrees of influence. Multiple measures may be used to represent observations expected if there were independence versus causal interaction among factors of interest. Such measures include measures that shall be described below, as well as other analyses focused on probabilistic lift over expectations with independence or deeper measures of causal influence. Because the user may want to control what metric is used and want to generate new metrics without having to re-run the pipeline end-to-end, the metrics calculation is online in one implementation.

Measures may include relative ratios (RR), which have been traditionally used in epidemiology to measure the ratio of the probability of an event (as revealed in a search or retrieval action used as a proxy for an experienced side effect in this case) in those searching for the drug versus those who did not. Another measure is described herein, namely the computation of the lift or ratio from the expected reporting with assumption of independent influences. This can be formalized as the divergence from independent causal events (DICE) score based on the divergence from expected reporting rate between the two variables given the assumption that they are independent. For example, given two drugs, for each symptom (S) in the spectra the system can compute expected reporting rate with independence assumption (ERRI):

$$E R R I = 1 - {\prod }_{i} ( 1 - {x}_{i} )$$

where x₁ is the proportion reporting S with drug one (1) only and x₂ is the proportion of reporting S with drug two (2) only.

To also consider the background probability of each of the symptoms, the equation above may include xₒ, the proportion reporting S with background or unobserved (or non-represented) influences only. The surveillance system can provide users with control over whether xₒ is included, depending on whether or not they wish the “background influence” to be considered. Additionally such factors as the overall influence of the cardinality of the distinct numbers of drugs or of drugs in a specific set of classes of medication may be considered as a distinct causal influence or represented as a modification of the ERRI equation in other ways.

Turning to drug-based examples, described are methods for the implicit identification of adverse drug events (AEs) and drug-drug interactions (DDIs) using large-scale behavioral data. Note that while search logs are used in one or more implementations, comprising the queries that users submit to search providers, it is understood that other sources of information such as social media also may have utility for this purpose e.g., to complement or replace the logs (although it is unclear how useful such sources may be since people may be less likely to discuss their medications in public forums than in private dialog with a search engine). These data are available to search providers in large quantities, enabling the development of a sensor network for identifying signals of public health information regarding medications, medical devices and/or procedures. Salient signals mined from this network can be used to inform focused follow-up investigations, including clinical trials, by organizations such as the FDA and pharmaceutical companies.

In any event, the large volume of search data may be on the order of millions or billions of medical queries from which to monitor medication-related interests. Still other sources such as surveys (including self-reports of adverse events provided by drug consumers) and electronic medical records also may be mined for this purpose, although strictly speaking these are not behavioral sources, they are considered behavioral sources for purposes of their usage herein.

The system exemplified in FIG. 1 may be used by analysts in government or industry to identify avenues for further exploration, or by consumers curious about potential interactions for the drugs they have been prescribed by physicians.

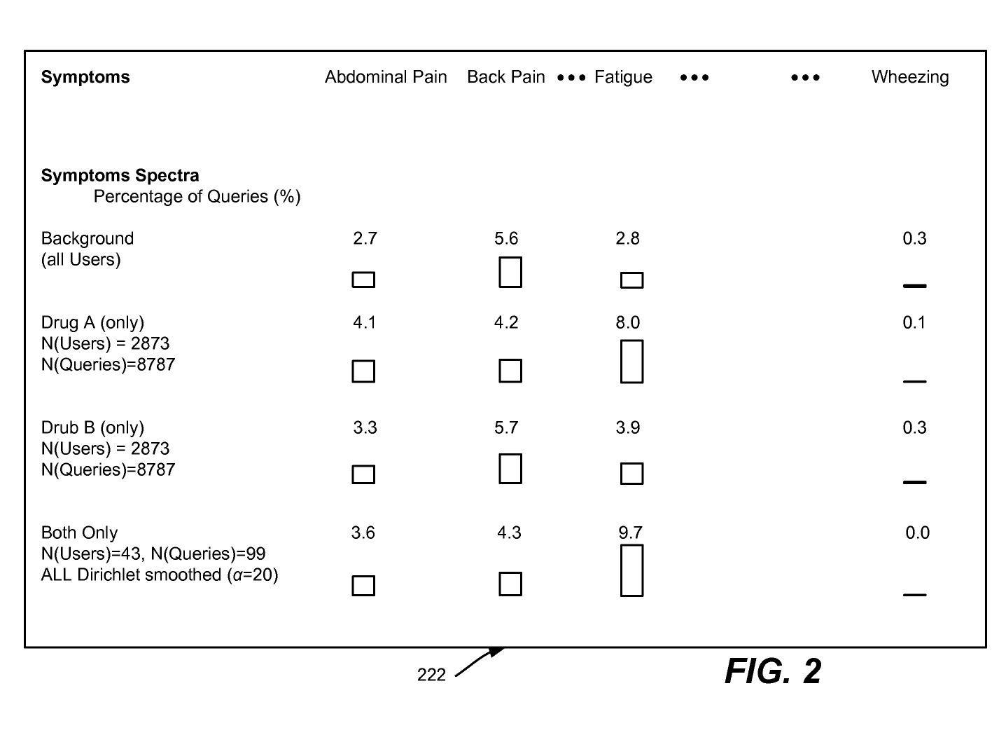

FIG. 2 shows an example partial screenshot (e.g., rendered via a browser) from the system related to the computation of symptom spectra 222. More particularly, looking for the presence of particular symptoms associated with a particular condition (e.g., hyperglycemia) is one task; broadening the analysis beyond a particular drug-pair and symptom set of interest is a different task because of the lack of any robust list of possible outcomes to use in probing the log data. To address this, described herein is symptom spectra, comprising a probability distribution across a standard set of symptoms (e.g., a set of around one hundred common symptoms) computed using different groups of users. Note that FIG. 2 is a visible representation of symptom spectra data, and only shows four example symptoms and their probability distributions; however it is understood that an actual user interface rendering may show on the order of one-hundred symptoms, and that various color schemes and the like may be used to more easily differentiate among the represented data.

As also shown in FIG. 2, groups of users may include (a) all users independent of whether they search for any of the medications of interest, (b) those users observed searching for a fixed number of unique drugs irrespective of the drugs of interest (where drug cardinality is defined as a way to infer more information about health status, as described below), (c) those users searching for each of the N drugs of interest, and (d) those users searching for all of the N drugs of interest.

As can be seen in FIG. 2, there are differences in the distributions in the spectra. For example, the likelihood of fatigue is higher in those groups who only search for both drugs.

Given these spectra, the system is able to compute the significance of the interaction between the drugs and the symptoms, by considering changes in the symptom spectra for users in the different groups of interest (e.g., those who query for the two drugs of interest independently and those who query for both drugs). An analyst also may visibly see these data. If the differences between both single-drug spectra and the paired-drug spectrum are statistically significant, e.g., using one of many appropriate statistical methods, such as including a two-sample Kolmogorov-Smirnov Test, then the system may conclude that there may be an interaction between the drugs. There may be other factors contributing to the interaction, and thus care needs to be taken to consider the symptoms that may be leading to the use of the medication in the first place (these are not caused by the medication), as well as those that would be expected from users with similar demographic attributes (e.g., age range, gender) to those likely to be prescribed the medication.

To address issues with data sparseness (low user and/or query counts) for a particular symptom spectrum, steps may be taken to smooth the spectrum with respect to background distribution (e.g., using Dirichlet smoothing). This may be made visible to users in the symptoms spectra representation 222.

In addition to relative ratios and the divergence from independent causal events, there are other ways to compute the background influence that can help consider factors such as users' health status. For example, the drug cardinality may be modeled, which is the total number of unique drugs searched for by those users from whom the background distribution is calculated. Cardinality may be used to model unknown hidden processes, such as demographic information (e.g., assuming that older people typically take more drugs) or general health and wellbeing (e.g., assuming that more medications means more ailments). With a factor h (for health status), that yields Hᵢ=hᵢ(n) for each symptom class, where n is the drug cardinality. Note that in the computation of cardinality the system may not want to include drugs from the same class that substitute for one another, (e.g., two drugs both may be proton pump inhibitors and taken for the same reason). Category information may be extracted from drug databases (e.g., for some drug A, the categories may be “Bronchodilator Agents”, “Adrenergic beta-Agonists” and “Sympathomimetic”). Drugs that share at least one category may be regarded as being substitutable and are therefore combinable in one analysis.

Given a cardinality influence score for each symptom S (grouped together with its associated synonyms), the system can use that as a substitute for the background, or alternatively compute the independent influence. For example, for cardinality 2, compute with just xₒ, the plain background without cardinality factor (hₛ(0)=xₒ), and when considering the health status, hₛ(2), compute the distribution across all users querying for exactly two of the drugs in the set. The system may then use that number as an input to the larger computation for each symptom set instead of the standard background value described above.

One challenge is determining which cardinality value is most appropriate for a given drug combination. Users may be provided with control over this, but this may vary between drugs and not be immediately clear. One way to do this is to assume that the cardinality value reflects some aspects of searchers' health status, whereby the system may select the most appropriate cardinality value for a given drug pair by considering the characteristics of users who are typically prescribed this medication; (e.g., if those prescribed the medication are typically older, or often have other concurrent conditions, then a high cardinality value may be appropriate).

Multiple drugs may be visually and algorithmically compared. For example, given that in one scenario a user may want to compare many pairs of drugs (e.g., compare all pairs of the Top N best-selling drugs in US, compare M new drugs with N existing drugs and so on), one or more implementations of the system provide a way to visualize the comparison so that analysts and other consumers can attain an overview of the interactions.

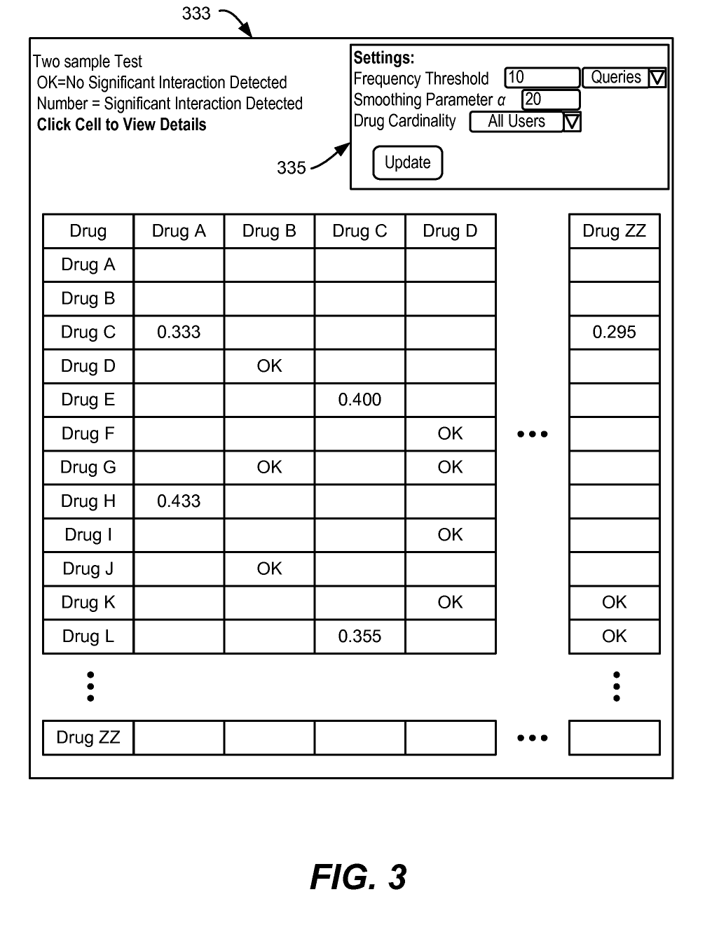

One way to visualize this is via an N×M matrix, with each cell representing a drug pair. FIG. 3 represents a partial screenshot example 333 from one implementation of the system. The contents of each cell depict the presence/absence of an interaction, if determinable, and the strength of the interaction (if there is one). Other visualizations also may be made available. As can be seen, the user is able to interact via interface elements in block 335.

Because the number of prescribed drugs is large, it may be computationally infeasible to compare all pairs, triples, and so on. The computational complexity increases exponentially with the addition of each new dimension representing a new set of drugs to be considered. To address this, possible combinations may be filtered to only those of interest based on their popularity (quantifying the extent of the likely benefit of an interaction discovery, which may be determined from the number of queries as well as sales information). Alternatively, (or in addition to filtering), the system may handle the combinatorial complexity as well as low counts for some drugs, by using drug classes rather than the specific drug names. This provides a coarse-grained way to narrow the analysis to particular classes of interest, which may then be explored more in follow-up studies.

Turning to temporal relationships and causality, the examples provided thus far assume that all of the data is available in the time span of the logs. However, more accurate detection of interaction effects may be possible if considering the temporal relationship between the emergence of the drug search and the symptom search (e.g., drug→side-effect may be a stronger signal than side-effect→drug). However, these are logs are unable to discern when a user started/terminated consumption of the medication, and when he or she started experiencing side effects. This makes it challenging to develop causal models that can accurately capture changes in searchers' health state over time. Symptoms may arise later in time after some use; further and also people may first input their queries only when symptoms they suspect are related appear. The amount of time between the drug and the symptom search needed to make an inference about causality (e.g., same query, same day, one week, one-month) may be varied in this analysis. For a recently-released drug, there may be a correlation between its introduction to the public and when queries start appearing.

The analysis described above focuses on the overall presence and absence of the symptom within a certain time window of the drug appearing, most likely following the first occurrence of the drug in the timespan of the searchers' log. The system also may consider the time series of drug and symptom searching for each of the users, and align those on the first occurrence of the drug in the logs. The alignment is beneficial in that it increases the amount of data available; rather than using sparse time series from each user, there is an aggregated temporal distribution from all searchers. Given this alignment, the system may employ more sophisticated causal modeling methods such as Granger causality to compute the likelihood of symptoms being reliably forecast by drug searching, and help move from correlation to causal influences.

As well as watching for the emergence of drugs within a user over time and mining associated side effects, the system may consider cases where side effects may be associated with people withdrawing from particular medications (e.g., skipping heartbeat coming off a steroid). This is based upon the detection of cases where users are less or no longer interested in a particular medication, observable by reduced query volume for that drug or a complete cessation of searching for it. This may be more challenging to interpret than presence of the drug in the logs, because users may stop querying for a number of reasons (e.g., they know a sufficient amount about the drug), only one of which is related to terminating its use.

Characteristics of the users' search queries may be used to provide information to build better quality user cohorts and enable finer-grained analysis of drugs. For example, the frequency of drug searching within a user might signal different interests or intent. In the analysis above, every drug search within a user counts once, but some people will search multiple times and may be treated differently in the analysis (e.g., frequent drug searching may suggest severe health concerns or that the user is a medical professional). Content of other queries from the searcher may be used to estimate demographics, as gender and age affect interests (as well as who tends to use the internet more) and therefore search behavior. Background levels of information seeking on symptoms, and for background levels conditioned on specified factors, such as searches on terms (e.g., symptoms, diseases, medications), topical classes of searches, sessions, such pattern evidence in search behavior such as the burstiness of information seeking, abruptness of search terms/topics, and so forth also may be considered.

As set forth above, although the examples were directed to the effect of medications, it is understood that similar analyses may be applied to other related searching in the medical domain (and beyond). For example, the technology may automatically detect, by the monitoring of medical device searching, complications that may be associated with medical device use, e.g., the use of CPAP machines and the development of tinnitus (ringing in the ears). Similarly, medical procedures that may have unintended consequences may be detected.

In addition to adverse effects, beneficial effects may be discovered. For example, users searching on a medication (as a proxy for taking a certain drug) that has nothing to do with allergies may start querying for why their allergies have disappeared or lessened.

Thus, discovering side effects, rather than confirming effects, involves the comparison of many combinations of drugs and exploring their impact on the medical symptoms (if any) that people experience from their consumption. Aspects include computing scores for recognizing signals in data, including various measures of surprising or anomalous “under-querying” or “over-querying.” Such scores may include divergence from independence and disproportionality analysis, for single and multiple attributes (e.g., single symptom class or multiple symptom classes). This may include developing characterizations of signal and error via use of knowns or ground truth data. Such scores may include the development and application of independence analysis (the DICE and DICE Ratio), including the incorporation of background information from users, users of particular drug cardinalities (e.g., the number of drugs that they search for, suggesting a particular health status), symptoms for which the drug is usually prescribed (or people in the prescribed cohort may experience independent of medications given factors such as age, gender, and so forth), and already-known side effects of drugs (which are removed/highlighted in analysis).

Aspects also include the construction and application of symptom spectra to support the detection of anomalous or surprising changes in symptoms within a cohort of users searching for a particular drug or drug tuple (versus background). Also described are models for understanding background or “prior” processes, such as background levels of information seeking on symptoms, and for background levels conditioned on specified factors, such as searches on terms (e.g., symptoms, diseases, medications), topical classes of searches, sessions, such pattern evidence in search behavior such as the burstiness of information seeking, abruptness of search terms/topics, etc., classifiers that provide probability distributions about user goals or cohorts (e.g., inferences about gender, age, other demographics). Reasoning about user cohorts is provided, including cohort demographics, e.g., age and gender effects using methods such as drug cardinality and query content analysis.

Models may seek to identify causality among influences seen via search logs, e.g., using temporal dependencies, such as when the drug query precedes the symptom query, or more sophisticated methods such as Granger causation. The system may model the emergence of drug side effects over prolonged periods, including changes in the frequency of drug searching within a user over time. Other models for combining inferred influences of independent identified “factors” may be provided, such as of multiple drugs seen in information seeking on medications. Still further, multi-drug interactions beyond pairs of drugs to consider N-way analyses of medications may be provided.

Other models may include probabilistic models for explaining influences and dependences among users and informational goals over time, and models for capturing the influence of external factors and for correcting for external factors, such as news media on information seeking, including use of data from logs of news stories, behavioral data on interaction with news e.g., via logs, measures of trending of interests via e.g., using time series modeling methods. Techniques may capture key clinical correlates of searching, browsing, posting, and other social media.

Methods may be employed that identify via queries and patterns of queries and page retrievals within sessions and over longer time periods to identify disorders and symptoms that are likely to be input as exploratory versus as experiential during information gathering where the nature and temporal structure of queries and retrieval provide evidence of symptoms or disorders being experienced by searchers themselves. For example, a searcher exploring web content on a medication may search on the medication and also explore details on the constellation of side effects as described on web pages on this medication. Such views of the timing of access of queries on the medication with access of information with queries on multiple of the symptoms listed may indicate exploration of multiple aspects of engaging with a new medication. Behaviors associated with experienced health outcomes may look quite distinct to such exploratory interaction. For example, a log may reveal that an index event, that a user has searched on a medication with an intensive search, followed by little attention over a period of time to the medication. Later, signs of interest and curiosity about gradually worsening symptoms may be revealed in a search logs, reflecting the natural history of symptoms, e.g., associated with a worsening gastric bleed, evolving from the darkening of stools to the coughing up of dark, “coffee ground” like material. The temporal disconnection of the searches on terms indicating gradually worsening symptoms of a certain type from the initial index event that provides evidence that a searcher is taking a medication can be used to identify the likelihood of experiential versus solely exploratory search on disorders and symptomotology.

The evidence may be processed to make predictions of future outcomes from the evidence. For example, evidence may be used to learn about and make predictions regarding the appearance or progression of disorders and/or symptoms.

Signals from multiple logs and sources may be integrated and fused, including logs of behavioral data across search and browsing, communication social media, surveys, electronic medical records and so forth.

New applications may result from data based upon the complications of medical devices (or other devices), the comorbidities of disease as revealed in search or retrieval, within time and across time—per evolution of syndromes (e.g., snoring→apnea, CPAP→high blood pressure, myocardial infarction, and so forth).

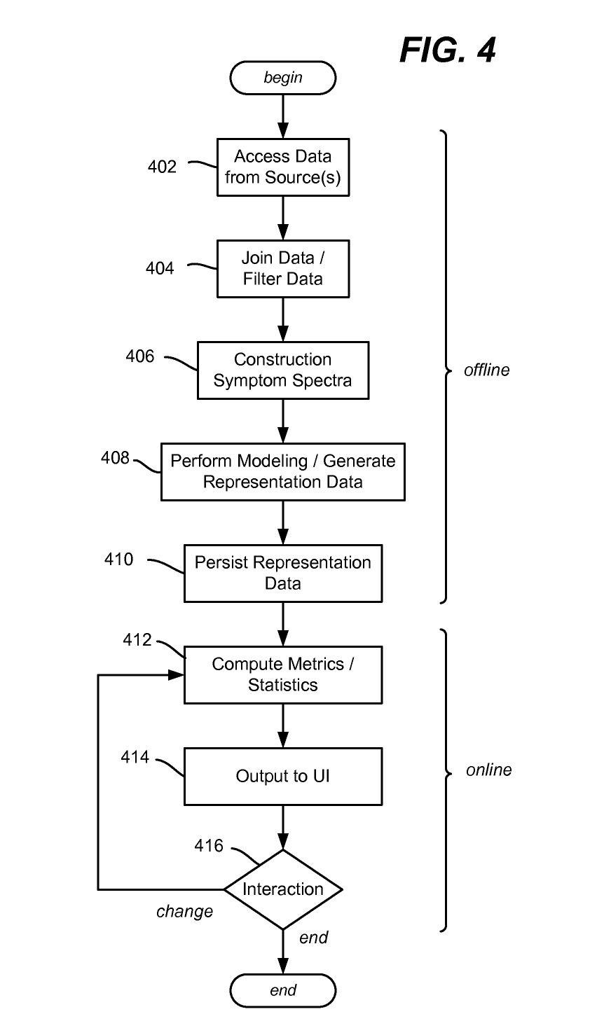

FIG. 4 is a flow diagram summarizing some of the example operations/steps described herein, beginning at step 402 where the behavioral data of one or more sources are accessed. Step 404 represents joining the data (if there are multiple sources) and any filtering of the data, e.g., to combine classes, for example.

Step 406 represents constructing the symptom spectra. As described above, this is any suitable arrangement of data that relates symptoms to user queries, informational posts and so forth.

Step 408 represents performing any modeling and so forth to generate the representation data. Note that steps 406 and 408 may work together, e.g., modeling as described herein may be used to an extent in constructing the symptom spectra. Step 410 persists the representation data.

Steps 412, 414 and 416 represent the online operation, including computing the metrics and statistics as needed, and outputting the desired rendering to the UI (e.g., dashboard). Interaction may change the computation/rendering as needed based upon user input.

### Example Networked and Distributed Environments

One of ordinary skill in the art can appreciate that the various embodiments and methods described herein can be implemented in connection with any computer or other client or server device, which can be deployed as part of a computer network or in a distributed computing environment, and can be connected to any kind of data store or stores. In this regard, the various embodiments described herein can be implemented in any computer system or environment having any number of memory or storage units, and any number of applications and processes occurring across any number of storage units. This includes, but is not limited to, an environment with server computers and client computers deployed in a network environment or a distributed computing environment, having remote or local storage.

Distributed computing provides sharing of computer resources and services by communicative exchange among computing devices and systems. These resources and services include the exchange of information, cache storage and disk storage for objects, such as files. These resources and services also include the sharing of processing power across multiple processing units for load balancing, expansion of resources, specialization of processing, and the like. Distributed computing takes advantage of network connectivity, allowing clients to leverage their collective power to benefit the entire enterprise. In this regard, a variety of devices may have applications, objects or resources that may participate in the resource management mechanisms as described for various embodiments of the subject disclosure.

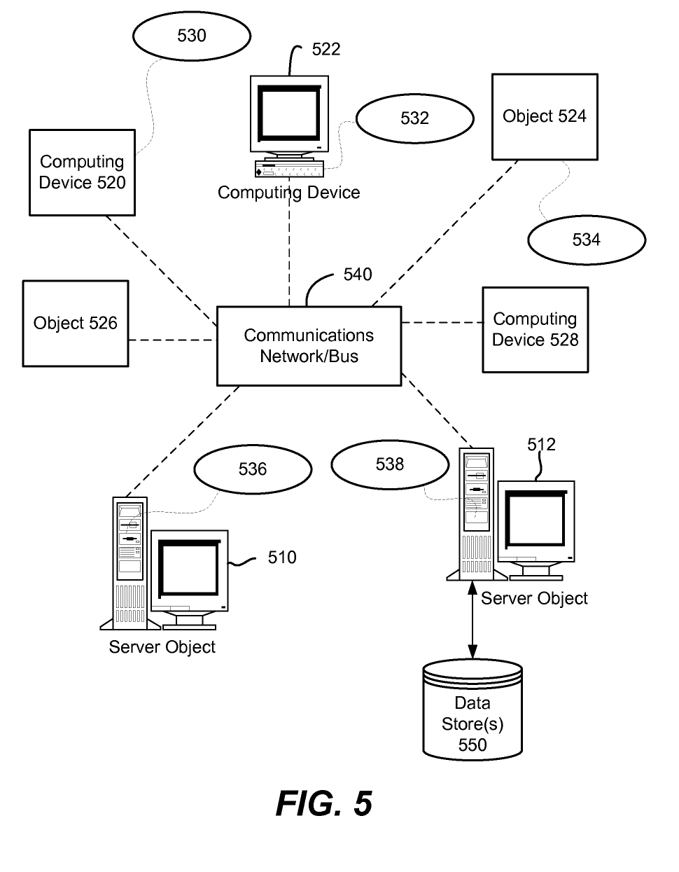

FIG. 5 provides a schematic diagram of an example networked or distributed computing environment. The distributed computing environment comprises computing objects 510, 512, etc., and computing objects or devices 520, 522, 524, 526, 528, etc., which may include programs, methods, data stores, programmable logic, etc. as represented by example applications 530, 532, 534, 536, 538. It can be appreciated that computing objects 510, 512, etc. and computing objects or devices 520, 522, 524, 526, 528, etc. may comprise different devices, such as personal digital assistants (PDAs), audio/video devices, mobile phones, MP3 players, personal computers, laptops, etc.

Each computing object 510, 512, etc. and computing objects or devices 520, 522, 524, 526, 528, etc. can communicate with one or more other computing objects 510, 512, etc. and computing objects or devices 520, 522, 524, 526, 528, etc. by way of the communications network 540, either directly or indirectly. Even though illustrated as a single element in FIG. 5, communications network 540 may comprise other computing objects and computing devices that provide services to the system of FIG. 5, and/or may represent multiple interconnected networks, which are not shown. Each computing object 510, 512, etc. or computing object or device 520, 522, 524, 526, 528, etc. can also contain an application, such as applications 530, 532, 534, 536, 538, that might make use of an API, or other object, software, firmware and/or hardware, suitable for communication with or implementation of the application provided in accordance with various embodiments of the subject disclosure.

There are a variety of systems, components, and network configurations that support distributed computing environments. For example, computing systems can be connected together by wired or wireless systems, by local networks or widely distributed networks. Currently, many networks are coupled to the Internet, which provides an infrastructure for widely distributed computing and encompasses many different networks, though any network infrastructure can be used for example communications made incident to the systems as described in various embodiments.

Thus, a host of network topologies and network infrastructures, such as client/server, peer-to-peer, or hybrid architectures, can be utilized. The “client” is a member of a class or group that uses the services of another class or group to which it is not related. A client can be a process, e.g., roughly a set of instructions or tasks, that requests a service provided by another program or process. The client process utilizes the requested service without having to “know” any working details about the other program or the service itself.

In a client/server architecture, particularly a networked system, a client is usually a computer that accesses shared network resources provided by another computer, e.g., a server. In the illustration of FIG. 5, as a non-limiting example, computing objects or devices 520, 522, 524, 526, 528, etc. can be thought of as clients and computing objects 510, 512, etc. can be thought of as servers where computing objects 510, 512, etc., acting as servers provide data services, such as receiving data from client computing objects or devices 520, 522, 524, 526, 528, etc., storing of data, processing of data, transmitting data to client computing objects or devices 520, 522, 524, 526, 528, etc., although any computer can be considered a client, a server, or both, depending on the circumstances.

A server is typically a remote computer system accessible over a remote or local network, such as the Internet or wireless network infrastructures. The client process may be active in a first computer system, and the server process may be active in a second computer system, communicating with one another over a communications medium, thus providing distributed functionality and allowing multiple clients to take advantage of the information-gathering capabilities of the server.

In a network environment in which the communications network 540 or bus is the Internet, for example, the computing objects 510, 512, etc. can be Web servers with which other computing objects or devices 520, 522, 524, 526, 528, etc. communicate via any of a number of known protocols, such as the hypertext transfer protocol (HTTP). Computing objects 510, 512, etc. acting as servers may also serve as clients, e.g., computing objects or devices 520, 522, 524, 526, 528, etc., as may be characteristic of a distributed computing environment.

### Example Computing Device

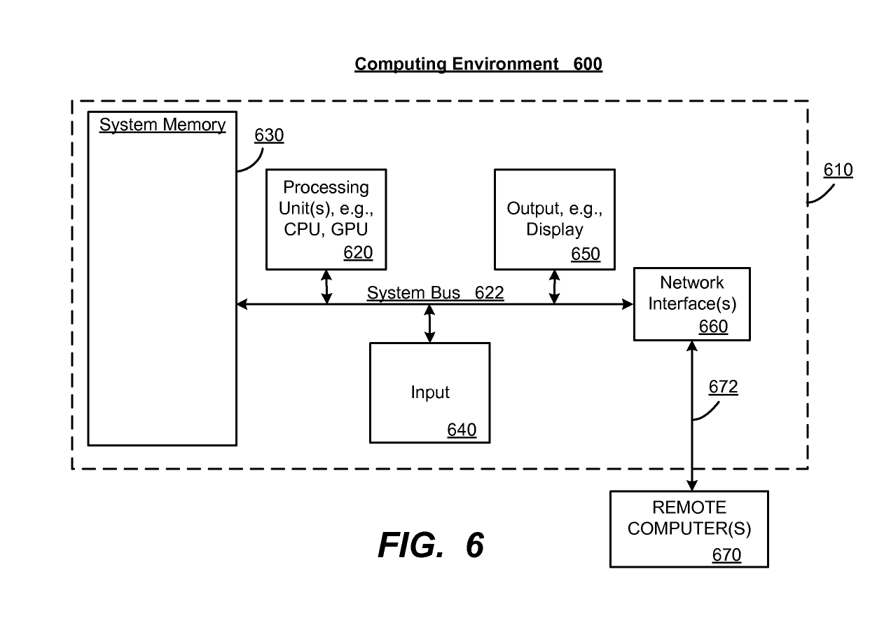

As mentioned, advantageously, the techniques described herein can be applied to any device. It can be understood, therefore, that handheld, portable and other computing devices and computing objects of all kinds are contemplated for use in connection with the various embodiments. Accordingly, the below general purpose remote computer described below in FIG. 6 is but one example of a computing device.

Embodiments can partly be implemented via an operating system, for use by a developer of services for a device or object, and/or included within application software that operates to perform one or more functional aspects of the various embodiments described herein. Software may be described in the general context of computer executable instructions, such as program modules, being executed by one or more computers, such as client workstations, servers or other devices. Those skilled in the art will appreciate that computer systems have a variety of configurations and protocols that can be used to communicate data, and thus, no particular configuration or protocol is considered limiting.

FIG. 6 thus illustrates an example of a suitable computing system environment 600 in which one or aspects of the embodiments described herein can be implemented, although as made clear above, the computing system environment 600 is only one example of a suitable computing environment and is not intended to suggest any limitation as to scope of use or functionality. In addition, the computing system environment 600 is not intended to be interpreted as having any dependency relating to any one or combination of components illustrated in the example computing system environment 600.

With reference to FIG. 6, an example remote device for implementing one or more embodiments includes a general purpose computing device in the form of a computer 610. Components of computer 610 may include, but are not limited to, a processing unit 620, a system memory 630, and a system bus 622 that couples various system components including the system memory to the processing unit 620.

Computer 610 typically includes a variety of computer readable media and can be any available media that can be accessed by computer 610. The system memory 630 may include computer storage media in the form of volatile and/or nonvolatile memory such as read only memory (ROM) and/or random access memory (RAM). By way of example, and not limitation, system memory 630 may also include an operating system, application programs, other program modules, and program data.

A user can enter commands and information into the computer 610 through input devices 640. A monitor or other type of display device is also connected to the system bus 622 via an interface, such as output interface 650. In addition to a monitor, computers can also include other peripheral output devices such as speakers and a printer, which may be connected through output interface 650.

The computer 610 may operate in a networked or distributed environment using logical connections to one or more other remote computers, such as remote computer 670. The remote computer 670 may be a personal computer, a server, a router, a network PC, a peer device or other common network node, or any other remote media consumption or transmission device, and may include any or all of the elements described above relative to the computer 610. The logical connections depicted in FIG. 6 include a network 672, such local area network (LAN) or a wide area network (WAN), but may also include other networks/buses. Such networking environments are commonplace in homes, offices, enterprise-wide computer networks, intranets and the Internet.

As mentioned above, while example embodiments have been described in connection with various computing devices and network architectures, the underlying concepts may be applied to any network system and any computing device or system in which it is desirable to improve efficiency of resource usage.

Also, there are multiple ways to implement the same or similar functionality, e.g., an appropriate API, tool kit, driver code, operating system, control, standalone or downloadable software object, etc. which enables applications and services to take advantage of the techniques provided herein. Thus, embodiments herein are contemplated from the standpoint of an API (or other software object), as well as from a software or hardware object that implements one or more embodiments as described herein. Thus, various embodiments described herein can have aspects that are wholly in hardware, partly in hardware and partly in software, as well as in software.

The word “exemplary” is used herein to mean serving as an example, instance, or illustration. For the avoidance of doubt, the subject matter disclosed herein is not limited by such examples. In addition, any aspect or design described herein as “exemplary” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used, for the avoidance of doubt, such terms are intended to be inclusive in a manner similar to the term “comprising” as an open transition word without precluding any additional or other elements when employed in a claim.

As mentioned, the various techniques described herein may be implemented in connection with hardware or software or, where appropriate, with a combination of both. As used herein, the terms “component,” “module,” “system” and the like are likewise intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on computer and the computer can be a component. One or more components may reside within a process and/or thread of execution and a component may be localized on one computer and/or distributed between two or more computers.

The aforementioned systems have been described with respect to interaction between several components. It can be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it can be noted that one or more components may be combined into a single component providing aggregate functionality or divided into several separate sub-components, and that any one or more middle layers, such as a management layer, may be provided to communicatively couple to such sub-components in order to provide integrated functionality. Any components described herein may also interact with one or more other components not specifically described herein but generally known by those of skill in the art.

In view of the example systems described herein, methodologies that may be implemented in accordance with the described subject matter can also be appreciated with reference to the flowcharts of the various figures. While for purposes of simplicity of explanation, the methodologies are shown and described as a series of blocks, it is to be understood and appreciated that the various embodiments are not limited by the order of the blocks, as some blocks may occur in different orders and/or concurrently with other blocks from what is depicted and described herein. Where non-sequential, or branched, flow is illustrated via flowchart, it can be appreciated that various other branches, flow paths, and orders of the blocks, may be implemented which achieve the same or a similar result. Moreover, some illustrated blocks are optional in implementing the methodologies described hereinafter.

### CONCLUSION

While the invention is susceptible to various modifications and alternative constructions, certain illustrated embodiments thereof are shown in the drawings and have been described above in detail. It should be understood, however, that there is no intention to limit the invention to the specific forms disclosed, but on the contrary, the intention is to cover all modifications, alternative constructions, and equivalents falling within the spirit and scope of the invention.

In addition to the various embodiments described herein, it is to be understood that other similar embodiments can be used or modifications and additions can be made to the described embodiment(s) for performing the same or equivalent function of the corresponding embodiment(s) without deviating therefrom. Still further, multiple processing chips or multiple devices can share the performance of one or more functions described herein, and similarly, storage can be effected across a plurality of devices. Accordingly, the invention is not to be limited to any single embodiment, but rather is to be construed in breadth, spirit and scope in accordance with the appended claims.

What is claimed is:

1. A method comprising, processing large-scale behavioral data to identify health-related effects in which a target outcome is unknown, including recognizing signals in the large-scale behavioral data, including detecting anomalous querying patterns, browsing activities, or both, and taking action upon detecting anomalous querying patterns, browsing activities, or both.

2. The method of claim 1 wherein processing the large-scale behavioral data comprises monitoring events related to one or more medications.

3. The method of claim 1 further comprising, determining based upon interaction behavior whether a set of querying patterns or browsing activities, or both, are indicative of exploratory or experiential information gathering.

4. The method of claim 1 wherein processing the large-scale behavioral data comprises monitoring events related to the use of one or more medical devices or procedures.

5. The method of claim 1 wherein taking action comprises computing significance of an interaction between a plurality of health-related events, including by considering changes between users in different groups of interests gathering information across a spectra.

6. The method of claim 1 wherein recognizing the signals in the large-scale behavioral data comprise monitoring querying patterns over time to determine time-related effects.

7. The method of claim 1 wherein processing the data includes constructing a dataset corresponding to of symptom spectra to detect anomalous or unexpected symptom-related data, or both, within a cohort of users.

8. The method of claim 1 wherein processing the data includes an independence analysis including incorporating at least one of: background information from users, users of particular drug cardinalities, symptoms for which a drug, device or procedure is usually prescribed, or already-known side effects.

9. The method of claim 1 wherein processing the data includes modeling at least one of: background processes, prior processes, background levels conditioned on specified factors, topical classes of searches, sessions, pattern evidence in search behavior, probability distributions regarding user goals or cohorts, user cohort reasoning, causality among influences seen via search logs, drug side effects over prolonged periods, combined inferred influences of independent identified factors, multi-drug interactions, or influences and dependences among users and informational goals over time.

10. The method of claim 1 wherein processing the data includes correcting for influence of news or trending interests, or both.

11. The method of claim 1 further comprising, predicting future outcomes regarding an appearance or progression of disorders or symptoms.

12. A system comprising, at least one processor and memory configured as an offline data processing subsystem, the subsystem configured to access behavioral data from one or more sources, and to generate spectra data based upon the behavioral data, the spectra data comprising a probability distribution across a set of data computed using different groups of users.

13. The system of claim 12 wherein the offline subsystem includes a join component configured to combine behavioral data from a plurality of sources.

14. The system of claim 12 wherein the offline subsystem includes a filter component configured to filter based upon at least one: symptoms, classes or drugs.

15. The system of claim 12 including a user interface configured to render a visible representation of medical-related entities and detected interactions of at least some of the entities.

16. The system of claim 12 wherein the different groups of users comprise at least two of: users who query for information on one medical entity, users who query for information on another medical entity, or users who query for information on two or more medical entities.

17. The system of claim 12 wherein the system includes an offline subsystem that generates representation data based at least in part upon the spectra data, and an online subsystem that provide a user interface for interacting with the representation data.

18. The system of claim 12 wherein the spectra data corresponds to symptoms or conditions, or both, related to at least one of: one or more drugs, one or more medical devices or one or more medical procedures.

19. One or more machine-readable storage media or logic having executable instructions, which when executed perform steps, comprising, generating a representation of behavioral data with respect to large scale sets of medical entity information, and processing the representation to recognize health-related effects of one or more medical-related entities based upon statistical analysis, in which a target outcome is unknown.

20. The one or more machine-readable storage media or logic of claim 19 wherein generating the representation of the behavioral data comprises generating a spectra.
